(19) United States
(12) Patent Application Publication
(10) Pub. No.: US 2008/0108863 A1
(43) Pub. Date: May 8, 2008

(54) WOMAN'S HEATING DEVICE FOR HEIGHTENING SEXUAL EXPERIENCE DURING COITUS

(76) Inventor: Karen H. Stephenson, Myrtle Beach, SC (US)

Correspondence Address: TILLMAN WRIGHT, PLLC, PO BOX 471581, CHARLOTTE, NC, 28247, US

(21) Appl. No.: 11/936,031

(22) Filed: Nov. 6, 2007

Related U.S. Application Data
(60) Provisional application No. 60/864,415, filed on Nov. 6, 2006.

(51) Int. Cl. A61F 5/00; A61F 7/00

(52) U.S. Cl. 600/38; 607/108; 607/114

(57) ABSTRACT

A method for enhancing sexual encounter of a woman during coitus includes heating a heat transfer pack that radiates heat; thereafter, warming the labia and clitoris of the woman by positioning the heat transfer pack in the pelvic region of a woman so that the heat transfer pack radiates heat from the pouch to the pelvic region of the woman, the positioning of the heat transfer pack including securing the heat transfer pack to the legs and waist of the woman; and once the clitoris and labia has been warmed, engaging in intercourse with the clitoris and labia in the warmed state. A method for facilitating the enhancement of a woman's sexual encounter during coitus includes the steps of selling a heating apparatus and, in conjunction therewith, providing appropriate instructions for use of the heating apparatus for the enhancement of a woman's sexual encounter during coitus.

### CROSS-REFERENCE TO RELATED APPLICATION

The present application is a U.S. nonprovisional patent application of, and claims priority under 35 U.S.C. §119(e) to, U.S. provisional patent application Ser. No. 60/864,415, filed Nov. 6, 2006, which provisional patent application is incorporated by reference herein.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### FIELD OF THE INVENTION

The present invention generally relates to devices and methods for enhancing sexual encounters. In particular, the present invention relates to a heating device and the use thereof for heightening a sexual experience, especially when the heating device is used by a woman prior to coitus.

### BACKGROUND OF THE INVENTION

The following patent references evidence that heat packs are well known for applying heating and cooling therapy to the human body.

Eatman U.S. Pat. No. 2,497,443 (“Eatman”) discloses a device for applying heat to the lower abdomen of a woman for alleviating periodic pain without interfering with the normal activities of the woman while the device is worn. The device comprises a V-shaped container made from a rigid material such as aluminum. The device includes a first belt that is secured to the upper ends of the V-shaped container and that is adapted to be secured about the waist of the wearer. The device further includes a second belt that is secured at the apex of the container and is adapted to also surround the body of the wearer below the waist, whereby the two belts together hold the container firmly against the body of the wearer. When worn, the container is oriented with the upper ends of the V-shaped container disposed above the apex of the container. This configuration is generally shown in FIG. 1 of Eatman. In order to heat the lower abdomen, the container is hollow and is adapted to receive and contain hot water. With respect to an alternative embodiment, Eatman discloses that, instead of being water-heated, the device could include an electrical heating element and battery.

Shelton U.S. Pat. No. 6,308,341 (“Shelton”) discloses the combination of an underpant and thermal treatment device for the application of cold or heat therapy and compression to the perineal, genital, and anal areas. More particularly, Shelton discloses an adjustable gel-pack which can apply both compression and thermal treatment to the perineal, genital, and/or anal areas for relief of pain and discomfort. The gel-pack includes connecting straps at each end and an underpant with loops or other attachment means on the front and back external surfaces of the underpant. The gel-pack, cooled or heated to a desired temperature, is placed in the genital, perineal, and/or anal areas outside the underpant. One connecting strap is attached to the back external surface of the underpant, and the other connecting strap is pulled forward with as much tension as desired and attached to the front external surface of the underpant, thereby providing temperature and compression treatment to the desired areas for relief of pain and discomfort. The gel-pack is retained in place by the underpant and, specifically, by a waistband that extends about the waist above the hips in an annular fashion that is sufficient to bear the tension produced by the gel-pack and connection straps.

U.S. Pat. No. 5,716,388 (“Petelle”) discloses a flexible fabric pouch for holding a heat transfer pack, the pouch including an adjustable strap for attaching the pouch to a selected part of a person's body for the application of thermal therapy. Petelle discloses that the pouch is preferably made up of a lightweight moisture absorbing fabric such as woven cotton or wool. Petelle further discloses that one side of the pouch may be formed of plural layers of material to provide greater insulation between the thermal pack and the wearer's body, whereby the pouch may be applied to the body in contact therewith on one or the other sides of the pouch depending on the rate of heat transfer desired between the pouch and the wearer's body.

U.S. Pat. No. 6,585,673 (“Bass”) discloses a heat pack positioning device which includes a pocket, a pair of elongated elasticized straps, and a pair of elongated extension straps. The straps are used for securing the heat pack to portion of a person's body as shown in FIGS. 14-17 of Bass.

U.S. Pat. No. 5,948,010 (“Adamec”) discloses a therapeutic heat application device for applying heat to various areas of a person's body. The device includes a pad portion having a wide central portion and a pair of opposed tapered side portions. The pad portion has a shape resembling a pair of lips, and the wide central portion is divided into a plurality of narrow sections by vertically extending seams. A pair of straps extend outwardly from the tapered side portions of the pad portion, and each of the straps have hook and loop fasteners disposed thereon for engaging each other when the straps wrap the pad portion around a body of a wearer. Each of the tapered side portions is divided into two sections by a horizontally extending seam. Adamec further discloses that the sections of the wide central portion and the tapered side portions are filled with cracked corn. Alternatively, Adamec discloses that other natural materials could be used in lieu of the cracked corn, such as dried beans, peas, rice, and even sand.

U.S. Patent Application Publication No. 2002/0193857 (“Lavine”) discloses a therapeutic device comprising an enclosed member and a cover. The cover encompasses the enclosed member and includes a slit for the insertion and removal of the enclosed member. When the enclosed member is removed, the cover can be washed to clean the cover. The enclosed member may comprise a bag and have a plurality of pieces of organic material therein that are capable of being exothermic when heated or endothermic when cooled. Lavine discloses that the bag is preferably filled with feed and/or seed corn, but that the bag alternatively can be filled with rice, cherry pits, or any other seeds, grains and/or fruit pits capable of being exothermic when heated or endothermic when cooled. Lavine broadly discloses that the therapeutic device relieves pain, reduces stress, relaxes muscles, reduces swelling, or provides overall general relaxation and enjoyment.

U.S. Patent Application Publication No. 2006/0137072 (“Visco”) discloses a neck warmer that includes a microwaveable, heat-retaining material, such as buckwheat, rice, or the like.

U.S. Pat. Nos. 5,948,010 and 5,584,086 (“VanWinkel”) discloses a therapeutic pillow used in treating pain. The pillow has a baglike cover filled with natural granular material having a water content in the range of 5% to 25% by weight and a preferred range of water content from 9% to 16%. VanWinkel also discloses that the therapeutic pillow can include different shapes and can be, for example, the shape of a toy for use by children, a pad shape with pockets for inserting hands or feet, or muff-shaped.

In addition to the foregoing, and while neither disclosed nor suggested by any of the foregoing patent references, it also is known to use warming massage oils and creams in the areas of the genitals of a woman for enhancing a woman's experience during coitus. The use of such oils and creams is believed to increase the chances of a woman experiencing an orgasm. One such cream, which is applied to the clitoris prior to intercourse, is sold under the trademark “FemElle,” further information about which can be found at http://www.femelle.com/sexualenhancement.html.

### SUMMARY OF THE INVENTION

The present invention includes many aspects and features. Moreover, while many aspects and features relate to, and are described in, the context of devices and methods for enhancing sexual encounters and, in particular, a heating device and the use thereof for heightening a sexual experience, especially when the heating device is used by a woman prior to coitus, it should be noted that the invention includes broad applicability and uses and is not intended to be limited by such description, the invention of a particular patent application being limited only by the claims of a patent granted there from.

In a first aspect of the invention, a heating device is used to enhance a sexual experience during coitus.

In another aspect of the invention, a heating device is specifically designed to be used in the pelvic region of a woman prior to coitus in order to enhance a sexual experience during coitus.

In a feature, the heating device includes a pouch and a heat transfer pack that is removably contained within the pouch.

In another feature, the heating device includes straps for securing a heat transfer pack to a pelvic region of woman.

In another aspect of the invention, a heating apparatus to be worn by a woman for the purposes of sexual enhancement during coitus includes: a pouch having an open configuration and a closed configuration, the pouch including a soft fabric exterior; a heat transfer pack that is removably contained within the pouch; and a positioning system configured to position the pouch having the heat transfer pack therein against the pelvic region of a woman for increasing blood flow to the woman's genitals and to warm the clitoris and labia, the positioning system configured to secure the pouch directly to each leg and to the waist of the woman.

In a feature, the positioning system includes, a first strap that is connected to the pouch and that is configured to extend between the legs of the woman and to extend around and encircle a first leg for securing the pouch directly to the first leg; a second strap that is connected to the pouch and that is configured to extend between the legs of the woman and to extend around and encircle a second leg for securing the pouch directly to the second leg; and a third strap that is connected to the pouch and that is configured to extend around and encircle the waist of the woman for securing the pouch directly to the waist. The straps may be elastically stretchable and/or adjustable. Additionally, a strap may include a first end fixed to the pouch on a first side thereof and a second end releasably connected to the pouch on a second, opposite side of the pouch; alternatively, a strap may include first and second strap components each having a proximal end and a distal end, with the proximal ends of the first and second strap components each fixed to the pouch and with the distal ends of the first and second strap components being releasably connected together. Still yet, the third strap may be wider than each of the first and second straps.

In another feature of this aspect, the pouch and the heat transfer pack contained therein are generally triangular in shape.

In another feature, the pouch at its widest edge is approximately eight inches in length and is approximately six inches in length from its widest edge to a bottom of the apex thereof, and the pouch, containing the heat transfer pack therein, is between about half of an inch to about an inch in thickness.

In another feature, both the pouch and the heat transfer pack contained therein are trapezoidal in shape.

In another feature, the pouch includes a soft fabric exterior.

In another feature, the pouch includes a soft fuzzy material around the perimeter thereof.

In another feature, the pouch includes ornamentation, including eyes, nose, and ears, such that the pouch resembles a cartoon face of a kitty cat.

In another feature, the heat transfer pack is filled with a natural granular material.

In another feature, the heat transfer pack is filled with rice.

In another feature, the heat transfer pack further comprises a tab for facilitating pulling and removing the heat transfer pack from the pouch when the pouch is in an open configuration.

In another feature, heat provided by the heat transfer pack results from an exothermic reaction.

In still yet another aspect, a method for enhancing sexual encounter of a woman during coitus includes heating a heat transfer pack that radiates heat; thereafter, warming the labia and clitoris of the woman by positioning the heat transfer pack in the pelvic region of a woman so that the heat transfer pack radiates heat from the pouch to the pelvic region of the woman, the positioning of the heat transfer pack including securing the heat transfer pack to the legs and waist of the woman; and once the clitoris and labia has been warmed, engaging in intercourse with the warmed clitoris and labia.

In a feature of this aspect, intercourse is performed without first removing the heat transfer pack.

In a feature of this aspect, intercourse is performed after repositioning of the heat transfer pack in the pelvic region of the woman.

In a feature of this aspect the heat transfer pack contains rice.

In a feature of this aspect, the method further includes engaging in foreplay during the warming of the labia and clitoris.

In a feature of this aspect, the heat transfer pack is generally triangular in shape.

In a feature of this aspect the heat transfer pack is trapezoidal in shape.

In a feature of this aspect, heat provided by the heat transfer pack results from an exothermic reaction.

Another aspect of the invention includes a method for facilitating the enhancement of a woman's sexual encounter during coitus and includes the steps of selling a heating apparatus and, in conjunction therewith, providing instructions for use of the heating apparatus for the enhancement of a woman's sexual encounter during coitus. The heating apparatus includes a heat transfer pack, and a positioning system configured to position the heat transfer pack against the pelvic region of a woman for warming the woman's clitoris and labia.

The instructions include the steps of heating the heat transfer pack; positioning the heat transfer pack in the pelvic region of a woman so that the heat transfer pack radiates heat to the external genitals of the woman; and engaging in intercourse with the woman's genitals warmed.

In a feature of this aspect, the positioning system configured to secure the pouch directly to each leg and to the waist of the woman.

In a feature of this aspect, heat provided by the heat transfer pack results from an exothermic reaction.

In a feature of this aspect, the heat transfer pack contains rice.

In another feature of one or more of the foregoing aspects, the positioning system comprises an undergarment including a loop material to which hook material is releasably attachable. The heat transfer pack in accordance with this feature includes hook material adhered thereto that is releasably attachable to the loop material of the undergarment. The undergarment preferably is fabricated, at least in part, from the loop material and the hook material is that commercially known and sold as “ykk” power hook material. The undergarment also preferably is of the boyshorts style panty and includes an opening through which the vulva—and in particular the clitoris and labia—are exposed. The loop material of the undergarment preferably surrounds the opening, and the heat transfer pack preferably includes the hook material adhered thereto along a periphery of the heat transfer pack in a configuration that corresponds to the opening in the undergarment. The garment further may include an area of loop material for repositioning of the heat transfer pack so that the heat transfer pack does not interfere with intercourse after warming of the clitoris and labia.

In an alternative embodiment in accordance with this feature, the undergarment is of the boyshorts style panty but does not include an opening through which the vulva are exposed; the loop material of the undergarment covers an area of the pelvic region; and the heat transfer pack includes hook material adhered to a corresponding surface area thereof for attachment to the loop material.

In addition to the aforementioned aspects and features of the present invention, it should be noted that the present invention further encompasses the various possible combinations of such aspects and features.

### BRIEF DESCRIPTION OF THE DRAWINGS

One or more preferred embodiments of the present invention now will be described in detail with reference to the accompanying drawings, wherein the same elements are referred to with the same reference numerals, and wherein:

FIG. 1 is front view of an embodiment of a heating device in accordance with one or more aspects of the present invention.

FIG. 2 is a back view of the heating device of FIG. 1.

FIG. 3 is a back view of the heating device of FIG. 1 with a flap of a pouch opened to expose a pack.

FIG. 4 is a back view of the heating device of FIG. 1 with the flap of the pouch opened and the pack removed from the pouch.

FIG. 5 is a close-up view of the pack of FIG. 4 removed from the pouch.

### DETAILED DESCRIPTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, one or more preferred embodiments of the present invention are next described. The following description of one or more preferred embodiments is merely exemplary in nature and is in no way intended to limit the invention, its application, or uses.

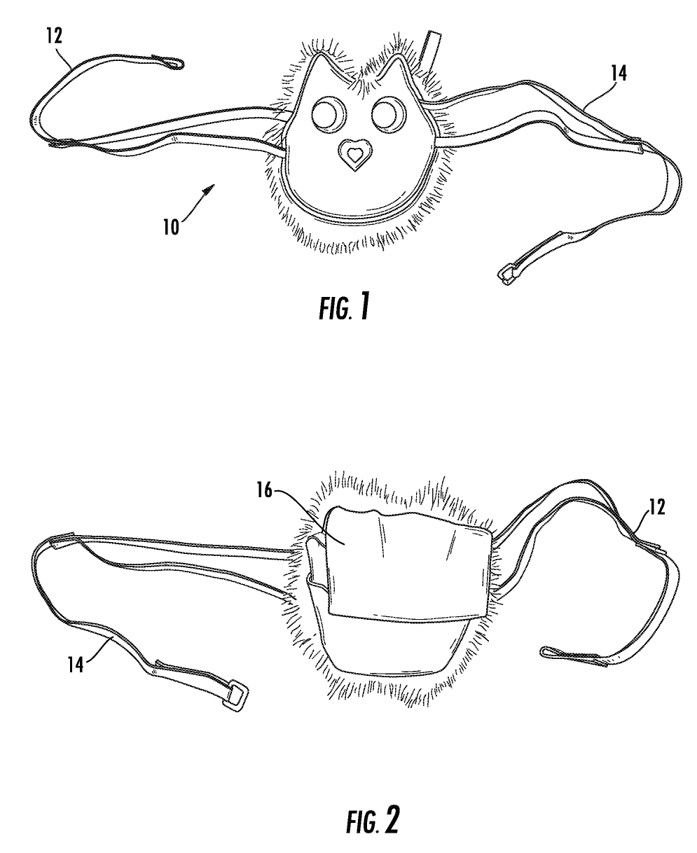

Turning now to FIGS. 1-5, a heating device generally indicated by the reference numeral 10 is illustrated. The heating device 10 is an original prototype of a heating device in accordance with an embodiment of the present invention. The heating device 10 is intended to be used by a woman for enhancing sexual experience during coitus.

As shown in FIG. 1, the heating device 10 includes a pouch and a strap. The pouch includes a soft fabric exterior and, as shown, includes a soft fuzzy material around the perimeter thereof. The pouch further includes ornamentation, including eyes, nose, and ears, such that the pouch resembles a cartoon face of a kitty cat. It is contemplated that a commercial embodiment of the heating device of the present invention will be sold under the trademark HOT KITTY or similar mark.

The strap includes a first strap component 12 and a second strap component 14. The first strap component 12 includes proximal ends that are attached to the pouch and, specifically, that are sewn to the pouch at two places along a first lateral side of the pouch.

Similarly, the second strap component 14 includes proximal ends that are attached to the pouch and, specifically, that are sewn to the pouch at two places along a second, opposite lateral side of the pouch.

The first strap component 12 also includes a distal, terminal end comprising a closed loop, and the second strap component 14 includes a distal, terminal end comprising a clear plastic clip that is dimensioned and configured to be received within the closed loop of the distal end of the first strap component 12, whereby the respective distal ends of the straps components 12,14 may be removably hooked together.

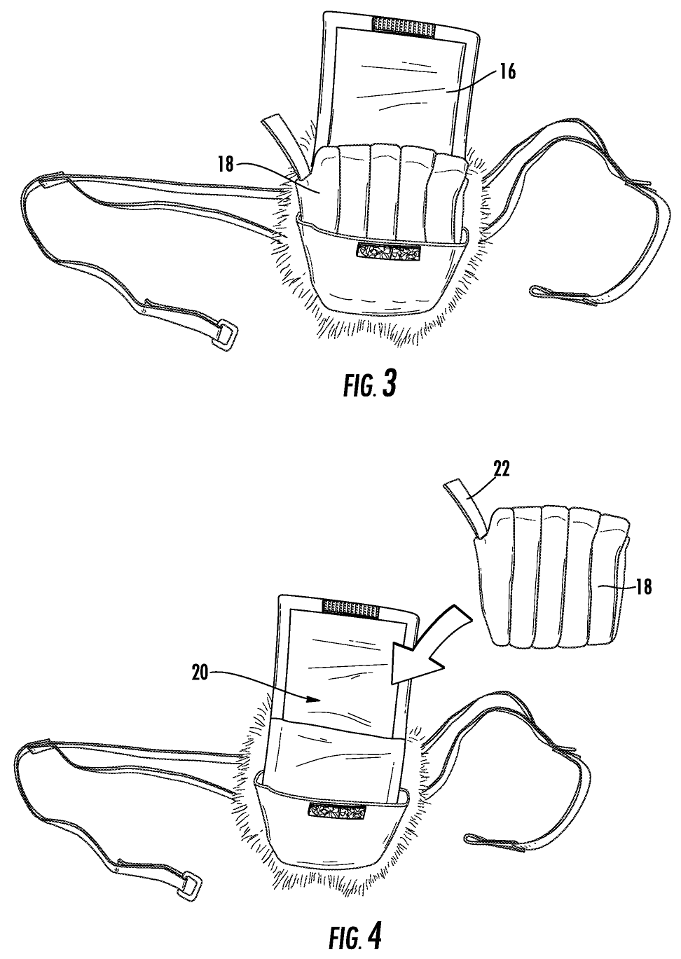

The pouch includes a flap 16, which is shown in FIG. 2 in a closed position. Conversely, the flap 16 is shown in an open position in FIG. 3. Hook-and-loop fasteners, such as those sold under the trademark VELCRO, are utilized in securing the flap 16 in the closed position, and such fasteners are illustrated in FIG. 3.

FIG. 3 further serves to illustrate that a pack 18 is contained within the pouch and is exposed when the flap 16 is in the open position. The pack 18 preferably is a heat transfer pack. Furthermore, the heat transfer pack 18 preferably is filled with a natural granular material and, specifically, the heat transfer pack 18 preferably is filled with rice. Also as shown in FIG. 3, the heat transfer pack 18 itself may include a tab 22 to facilitate pulling and removal of the heat transfer pack 18 from the pouch when the flap 16 is opened.

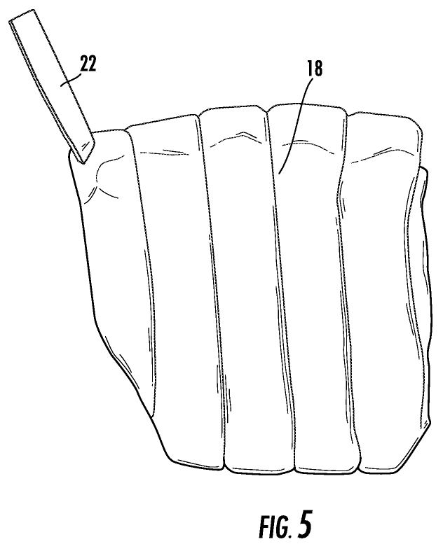

FIG. 4 illustrates the heating device 10 when the flap 16 is opened and the heat transfer pack 18 is removed from the pouch. A close-up view of the heat transfer pack 18 is illustrated in FIG. 5. As best shown in FIG. 5, the heat transfer pack 18 includes five ribbed sections, each of which preferably includes rice.

In operation, the heat transfer pack 18 is placed in a microwave oven and heated. The heat transfer pack 18 is heated for 1 or 2 minutes, after which time the rice contained in the heat transfer pack 18 produces a moist heat. After heating in the microwave oven, the heat transfer pack 18 is then inserted into the pouch and the flap 16 is closed. The pouch then is secured in the pelvic region of the woman using the first and second strap components 12,14, wherein the heat transfer pack 18 radiates a warm moist heat from the pouch to the pelvic region of the woman. The pouch is secured by extending the strap components 12,14 around opposite respective sides of the body and joining the distal ends of the strap components 12,14 together in the area of the lower back of the woman. The pouch also preferably includes a soft exterior that creates a pleasurable tactile sensation when placed in the pelvic region of the woman.

The pouch preferably is left on the pelvic region for up to twenty minutes, during which time the heat will increase blood flow to the woman's genitals. Specifically, the heat from the heat pack will cause the vulva to warm and, in particular, the labia and clitoris to warm. It is believed that the increased blood flow will heighten sensitivity and enhance sexual encounter. The warm moist heat also is believed to have a relaxing and comforting effect that can be enjoyed during this time. Moreover, this period of time provides time for foreplay, if desired.

Following heating of the pelvic region, the heating device 10 is preferably removed or at least repositioned so that the heating pack 10 does not inhibit or otherwise interfere with coitus.

The heating device 10 is specifically designed to be worn by a woman for the purposes of sexual enhancement. However, it also is believed that, during coitus, a male partner also will experience a warming, pleasurable sensation with his penis upon penetration as a result of use of the heating device 10 of the present invention.

As will be appreciated, after use the heating pack 10 can be cleaned, if necessary, by removal of the heat transfer pack 18 and washing of the pouch, after which the pouch may be reused. The heat transfer pack similarly can be reused by simply reheating the pack in the microwave oven and reinserting the heat transfer pack into the pouch.

As further will be appreciated, use of the heating device 10 in accordance with an aspect of the present invention does not result in any oil stains or other mess requiring cleaning. Unlike oils and creams, the heating device 10 itself does not necessitate cleaning.

Heating devices in accordance with other embodiments of the present invention that are contemplated include modifications to the heating device 10 of FIGS. 1-5.

In accordance with a first such modification, both the pouch and the heat transfer pack contained therein are triangular in shape and are specifically dimensioned and configured to correspond to the generally triangular, pelvic area of a woman. In other words, the pouch and heat transfer pack are “bikini shaped.” In such a contemplated embodiment, it is anticipated that the pouch at its widest edge (i.e., the top edge) will be approximately eight inches in length and six inches in length from top to the bottom apex thereof. It is further anticipated that the pouch will be between about half of an inch to an inch in thickness (including the heat transfer pack contained therein).

In accordance with a second such modification, one or more of the straps 12,14 are adjustable and/or are elastically stretchable.

Still yet, in accordance with a third modification, the pouch includes straps that extend between the legs and around and encircle each leg for securing the heating device in the pelvic region. In this respect, one or more straps are secured to the pouch and extend between the legs and around and encircle a first leg for securing the pouch to the first leg, and one or more straps are secured to the pouch and extend between the legs and around and encircle a second leg for securing the pouch to the second leg.

In this modified heating device, one or more straps also may extend around the waist or hips, as with the heating device 10. Furthermore, the one or more straps extending around the waist may be wider than the straps extending around the and between the legs. Moreover, each strap may include two strap components having proximal ends that are attached to the pouch and distal ends that removable fasten to one another. Alternatively, each strap may include ends that fasten to the pouch itself, and each end of the strap may removably fasten to the pouch.

Based on the foregoing description, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

What is claimed is:

1. A heating apparatus to be worn by a woman for the purposes of sexual enhancement during coitus, comprising:
(a) a pouch having an open configuration and a closed configuration, the pouch including a soft fabric exterior;
(b) a heat transfer pack that is removably contained within the pouch; and
(c) a positioning system configured to position the pouch having the heat transfer pack therein against the pelvic region of a woman for increasing blood flow to the woman's genitals and to warm the clitoris and labia, the positioning system configured to secure the pouch directly to each leg and to the waist of the woman.

2. The heating apparatus of claim 1, wherein the positioning system includes,
(i) a first strap that is connected to the pouch and that is configured to extend between the legs of the woman and to extend around and encircle a first leg for securing the pouch directly to the first leg,
(ii) a second strap that is connected to the pouch and that is configured to extend between the legs of the woman and to extend around and encircle a second leg for securing the pouch directly to the second leg, and
(iii) a third strap that is connected to the pouch and that is configured to extend around and encircle the waist of the woman for securing the pouch directly to the waist.

3. The heating apparatus of claim 2, wherein the first, second and third straps are elastically stretchable.

4. The heating apparatus of claim 2, wherein the first, second and third straps are adjustable.

5. The heating apparatus of claim 2, wherein a strap includes a first end fixed to the pouch on a first side thereof and a second end releasably connected to the pouch on a second, opposite side thereof.

6. The heating apparatus of claim 2, wherein a strap includes first and second strap components each having a proximal end and a distal end, the proximal ends of the first and second strap components each fixed to the pouch and the distal ends of the first and second strap components being releasably connected together.

7. The heating apparatus of claim 2, wherein the third strap is wider than each of the first and second straps.

8. The heating apparatus of claim 1, wherein the pouch and the heat transfer pack contained therein are generally triangular in shape.

9. The heating apparatus of claim 8, wherein the pouch at its widest edge is approximately eight inches in length and is approximately six inches in length from its widest edge to a bottom of the apex thereof.

10. The heating apparatus of claim 9, wherein the pouch, containing the heat transfer pack therein, is between about half of an inch to about an inch in thickness.

11. The heating apparatus of claim 1, wherein both the pouch and the heat transfer pack contained therein are trapezoidal in shape.

12. The heating apparatus of claim 1, wherein the pouch includes a soft fabric exterior.

13. The heating apparatus of claim 1, wherein the pouch includes a soft fuzzy material around the perimeter thereof.

14. The heating apparatus of claim 1, wherein the pouch includes ornamentation, including eyes, nose, and ears, such that the pouch resembles a cartoon face of a kitty cat.

15. The heating apparatus of claim 1, wherein the heat transfer pack is filled with a natural granular material.

16. The heating apparatus of claim 1, wherein the heat transfer pack is filled with rice.

17. The heating apparatus of claim 1, wherein the heat transfer pack further comprises a tab for facilitating pulling and removing the heat transfer pack from the pouch when the pouch is in an open configuration.

18. The heating apparatus of claim 1, wherein heat provided by the heat transfer pack results from an exothermic reaction.

19. A method for enhancing sexual encounter of a woman during coitus, comprising:
(a) heating a heat transfer pack that radiates heat;
(b) after said step (a), warming the labia and clitoris of the woman by positioning the heat transfer pack in the pelvic region of a woman so that the heat transfer pack radiates heat to the pelvic region of the woman, the positioning of the heat transfer pack including securing the heat transfer pack to the legs and waist of the woman; and
(c) once the clitoris and labia has been warmed, engaging in intercourse with the clitoris and labia warmed.

20. The method of claim 18, wherein said step (c) is performed without removing the heat transfer pack.

21. The method of claim 18, wherein said step (c) is performed after repositioning of the heat transfer pack in the pelvic region of the woman.

22. The method of claim 18, wherein the heat transfer pack contains rice.

23. The method of claim 18, further comprising engaging in foreplay during the warming of the labia and clitoris.

24. The method of claim 18, wherein the heat transfer pack is generally triangular in shape.

25. The method of claim 18, wherein the heat transfer pack is trapezoidal in shape.

26. The method of claim 18, wherein heat provided by the heat transfer pack results from an exothermic reaction.

27. A method for facilitating the enhancement of a woman's sexual encounter during coitus, comprising the steps of
(a) selling a heating apparatus comprising,
(i) a heat transfer pack, and
(ii) a positioning system configured to position the heat transfer pack against the pelvic region of a woman for warming the woman's clitoris and labia;
(b) providing in conjunction with said step (a) instructions for use of the heating apparatus, the instructions including the steps of,
(i) heating the heat transfer pack, and
(ii) positioning the heat transfer pack in the pelvic region of a woman so that the heat transfer pack radiates heat to the external genitals of the woman, and
(iii) engaging in intercourse with the woman's genitals warmed.

28. The method of claim 27, wherein the positioning system is configured to secure the heat transfer pack directly to each leg and to the waist of the woman.

29. The method of claim 27, wherein the heat transfer pack contains rice.

30. The method of claim 27, wherein heat provided by the heat transfer pack results from an exothermic reaction.
